(19) United States
(12) Patent Application Publication
(10) Pub. No.: US 2025/0273348 A1
(43) Pub. Date: Aug. 28, 2025

(54) TECHNIQUES FOR HIERARCHICAL CLUSTERING WITH TIERED SPECIFICITY

(71) Applicant: OPTUM, INC., Minnetonka, MN (US)

(72) Inventor: Ryan Michael Allen, Onalaska, WI (US)

(21) Appl. No.: 18/589,994

(22) Filed: Feb. 28, 2024

(51) Int. Cl. G16H 50/70; G16H 20/00; G16H 50/20

(52) U.S. Cl. CPC G16H 50/70; G16H 20/00; G16H 50/20

(57) ABSTRACT

Techniques for hierarchical clustering with tiered specificity are disclosed herein. An example computer-implemented method includes receiving data points that each include data corresponding to a feature set. The example computer-implemented method further includes applying, a machine learning model to the data points to: cluster (i) a first portion of the data points into a first cluster set based on similarity values computed using a first subset of the feature set and (ii) a second portion of the plurality of data points into a second cluster set based on similarity values computed using a second subset of the feature set that is different from the first subset. The example computer-implemented method further includes generating a data object indicating a course of action for an entity associated with a first data point based on the first data point being included in the first cluster set or the second cluster set.

### TECHNICAL FIELD

The present disclosure generally relates to data clustering techniques and data object generation, and more particularly, to the use of hierarchical clustering with tiered specificity to facilitate generating data objects indicating courses of action for requesting entities.

### BACKGROUND

Distance-based clustering methods have found applications in a wide variety of domains due to their flexibility and capability to provide valuable insights. However, conventional distance-based clustering methods often struggle when clustering binary data and one-hot encoded data. For example, conventional methods configured to cluster categorical data (including binary data and/or one-hot encoded data) are often not hierarchical and provide no control over distance thresholds that determine how similar data should be to form a cluster. However, conventional hierarchical clustering techniques generally lack flexibility with respect to determining and/or implementing features that are to be considered when determining the various clustering tiers. Thus, for both non-hierarchical and hierarchical techniques, conventional clustering of binary data or one-hot encoded data can erroneously create (1) clusters with dissimilar and/or marginally similar data points and (2) significant amounts of “orphaned data” that fails to satisfy the clustering parameters and is thereafter needlessly excluded from interpretation.

Therefore, in general, creating a clustering method capable of accurately clustering binary data and one-hot encoded data is an area of great interest, and conventional techniques are insufficient for providing such accurate binary/one-hot encoded data clustering. Accordingly, a need exists for techniques that provide users with relevant data clusters that mitigate the negative effects stemming from a lack of accurate binary/one-hot encoded data clustering.

### SUMMARY

In some aspects, a computer-implemented method includes receiving, by one or more processors, a plurality of data points that each include data corresponding to a feature set, and applying, by the one or more processors, a machine learning model to the plurality of data points. Applying the machine learning model includes: clustering, based on similarity values computed using a first subset of the feature set, a first portion of the plurality of data points into a first cluster set, and clustering, based on similarity values computed using a second subset of the feature set that is different from the first subset, a second portion of the plurality of data points into a second cluster set. The computer-implemented method also includes generating, by the one or more processors, a data object indicating a course of action for an entity associated with a first data point based on the first data point being included in the first cluster set or the second cluster set.

In some aspects, a system includes memory and one or more processors communicatively coupled to the memory. The one or more processors are configured to receive a plurality of data points that each include data corresponding to a feature set, and apply a machine learning model to the plurality of data points. Applying the machine learning model includes: clustering, based on similarity values computed using a first subset of the feature set, a first portion of the plurality of data points into a first cluster set, and clustering, based on similarity values computed using a second subset of the feature set that is different from the first subset, a second portion of the plurality of data points into a second cluster set. The one or more processors are further configured to generate a data object indicating a course of action for an entity associated with a first data point based on the first data point being included in the first cluster set or the second cluster set.

In some aspects, one or more non-transitory computer-readable storage media include instructions that, when executed by one or more processors, cause the one or more processors to receive a plurality of data points that each include data corresponding to a feature set, and apply a machine learning model to the plurality of data points. Applying the machine learning model includes: clustering, based on similarity values computed using a first subset of the feature set, a first portion of the plurality of data points into a first cluster set, and clustering, based on similarity values computed using a second subset of the feature set that is different from the first subset, a second portion of the plurality of data points into a second cluster set. The instructions further cause the one or more processors to generate a data object indicating a course of action for an entity associated with a first data point based on the first data point being included in the first cluster set or the second cluster set.

### BRIEF DESCRIPTION OF THE DRAWINGS

The Figures described below depict preferred embodiments for purposes of illustration only. One skilled in the art will readily recognize from the following discussion that alternative embodiments of the systems and methods illustrated herein may be employed without departing from the principles of the disclosure described herein.

FIG. 1 depicts an example computing system in which various embodiments of the present disclosure may be implemented.

FIG. 2A depicts an example data point clustering and data object generation sequence, in accordance with various embodiments described herein.

FIG. 2B depicts an example cluster-based data object generation architecture, in accordance with various embodiments described herein.

FIG. 2C depicts an example data point clustering using different feature sets, in accordance with various embodiments described herein.

FIG. 3 depicts a flow diagram representing an example computer-implemented method, in accordance with various embodiments described herein.

### DETAILED DESCRIPTION

Broadly speaking, the techniques of the present disclosure relate to hierarchical, distance-based clustering algorithm(s)/model(s) configured to utilize similarity values (e.g., the Jaccard distance metric) and differing feature sets/subsets for different clustering tiers. The data points used as inputs to the clustering model each include data corresponding to a feature set (e.g., age, co-morbidities, gender). The clustering model is generally configured to cluster based on these similarity values computed using a first subset of the feature set, a first portion of the plurality of data points into a first cluster set; and cluster, based on similarity values computed using a second subset of the feature set that is different from the first subset, a second portion of the plurality of data points into a second cluster set. One or more processors of the systems described herein then generate a data object indicating a course of action for an entity associated with a first data point based on the first data point being included in the first cluster set or the second cluster set.

Thus, the techniques of the present disclosure hierarchically cluster data, in part, by utilizing different feature subsets at different clustering tiers. This is advantageous as not all data points included in a data set may satisfy the parameters (i.e., thresholds) applied across an entire feature space for inclusion in a cluster. Many data points may satisfy some, but not all, of the parameters across the entire feature space and may only satisfy all parameters when evaluated across a subset of the feature space. Importantly, clusters originating from clustering across such feature space subsets represent significant relationships between/among the clustered data points and yield valuable insights that are otherwise missed when such hierarchical clustering based on feature space subset(s) is not performed. As previously described, conventional hierarchical clustering techniques cluster data points across an entire, static feature space regardless of clustering tier. These conventional techniques thereby completely preclude the formation of clusters based on fewer than all features of the feature space, and correspondingly preclude the benefits of leveraging the relationships represented by such clusters. By contrast, the clustering algorithms described herein capture these significant relationships between/among data points by hierarchically clustering over different feature subsets at different clustering tiers, and therefore improve over conventional techniques that do not perform such clustering.

Further, conventional techniques often leave data points as orphaned data points because statically clustering across an entire feature space during different clustering tiers may not substantially impact the probability that an orphaned data point will be clustered at any tier. In these circumstances, the orphaned data points generally fail to provide significant insights, as it is difficult to derive trends from outcomes associated with such orphaned data points. However, the probability of any individual data point failing to satisfy the parameters (clustering criteria) for a subset of the feature space is less than the probability of the individual data point failing to satisfy the parameters for the entire feature space. The clustering algorithms of the present disclosure cycle through one or more clustering tiers while utilizing different subsets of the feature space. Thus, the clustering algorithms described herein may yield a compound probability of the individual data point failing to satisfy the parameters for each of the multiple subsets of the feature space that is significantly less than the probability of the individual data point failing to satisfy the parameters for the entire feature space. Accordingly, the clustering algorithms described herein avoid and/or reduce the number/frequency of orphaned data points by performing hierarchical clustering based on feature space subset(s).

Overall, the clustering algorithm(s)/model(s) described herein more accurately cluster data and improve data usability. More specifically, the clustering algorithm(s)/model(s) described herein hierarchically cluster data points by using different feature subsets of a feature space in different clustering tiers to generate clusters that more accurately reflect the relationships between/among data points than when using conventional techniques. The clustering algorithm(s)/model(s) described herein also improve data usability by reducing/eliminating orphaned data points through such feature space subset hierarchical clustering, thereby avoiding the loss of insights experienced by conventional techniques.

The techniques of the present disclosure also improve the functionality of a computing device (e.g., a hosting server such as a central server or a user computing device) at least by using a machine learning model in a particular way to enhance the intelligence or predictive ability of the computing device. This machine learning model, executing on the computing device, can more accurately cluster binary data and/or one-hot encoded data without generating significant orphaned data. That is, the present disclosure describes improvements in the functioning of the computer itself because the computing device can more accurately cluster binary data in a manner that improves resulting cluster-based data generation or decision making, such as generating (e.g., selecting) data objects (e.g., care paths) for users with similar symptoms, co-morbidities, demographic characteristics, etc. This improves over the prior art at least because existing systems are generally unable to accurately analyze and cluster such binary and/or one-hot encoded data to output predictive and/or otherwise generated responses designed to, e.g., improve a user's diagnostic/treatment efforts for illnesses based on the user's associated cluster(s).

Moreover, the present disclosure includes effecting a transformation or reduction of a particular article to a different state or thing, e.g., transforming or reducing the processing demand of a computing system (and associated subsystems/components/devices) from a non-optimal or error state to an optimal (or closer to optimal) state by eliminating irrelevant clusters, reducing orphaned data, generating more accurate care paths, and consequently substantially reducing redundant processing demand conventionally required to correct/change such erroneous actions/determinations.

Still further, the present disclosure includes specific features other than what is well-understood, routine, conventional activity in the field, or adding unconventional steps that demonstrate, in various embodiments, particular useful applications, e.g., applying, by the one or more processors, a machine learning model to the plurality of data points, wherein applying the machine learning model includes: clustering, based on similarity values computed using a first subset of the feature set, a first portion of the plurality of data points into a first cluster set, and clustering, based on similarity values computed using a second subset of the feature set that is different from the first subset, a second portion of the plurality of data points into a second cluster set; and generating, by the one or more processors, a data object indicating a course of action for an entity associated with a first data point based on the first data point being included in the first cluster set or the second cluster set, among others.

Of course, it should be appreciated that the advantages and technical improvements described above and elsewhere herein are not the only advantages and/or technical improvements that may be realized as a result of the techniques described herein. Other advantages and/or technical improvements to the functioning of a computer itself or other technologies or technical fields may be apparent to one of ordinary skill in the art.

As referenced herein, data used as input to the machine learning model is associated with various “features” indicating different categories for the values included as data points within the data (e.g., age, gender, co-morbidity, diagnosis code, service location). Further, “clusters” refer to groups of data the machine learning model determines to be substantially similar through evaluation/comparison of these features in combination with one or more parameters (e.g., a data point neighbor value, a distance threshold, a minimum cluster size). A “clustering tier” or “cluster tier” indicates a stage of the hierarchical clustering process performed by the machine learning model, wherein the features used to evaluate/compare data in different clustering tiers are different. Moreover, while described herein primarily in the medical context, the techniques described herein may be readily applied in any suitable field for any suitable purpose.

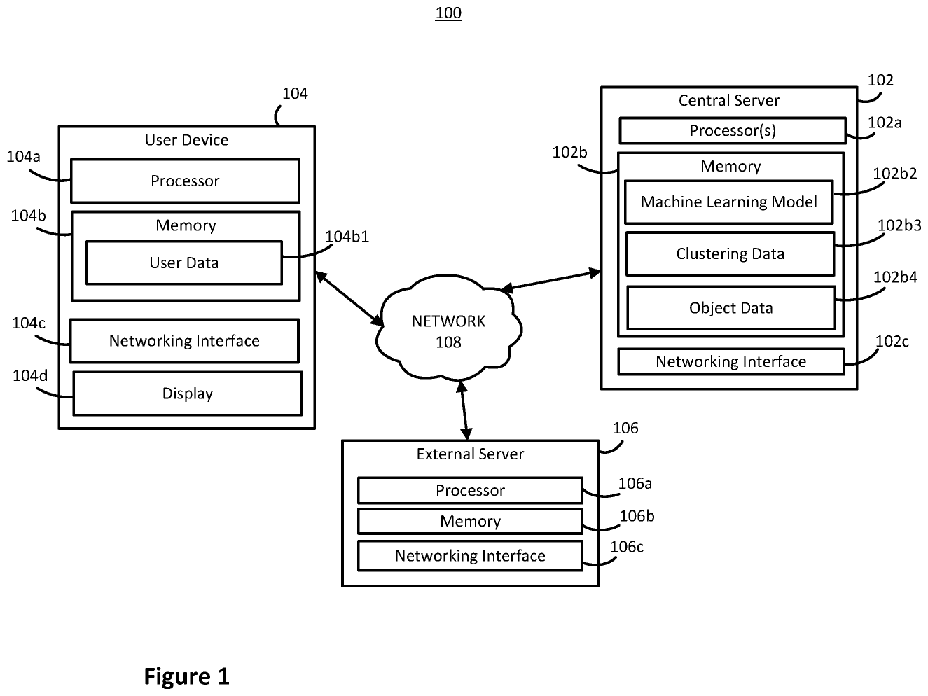

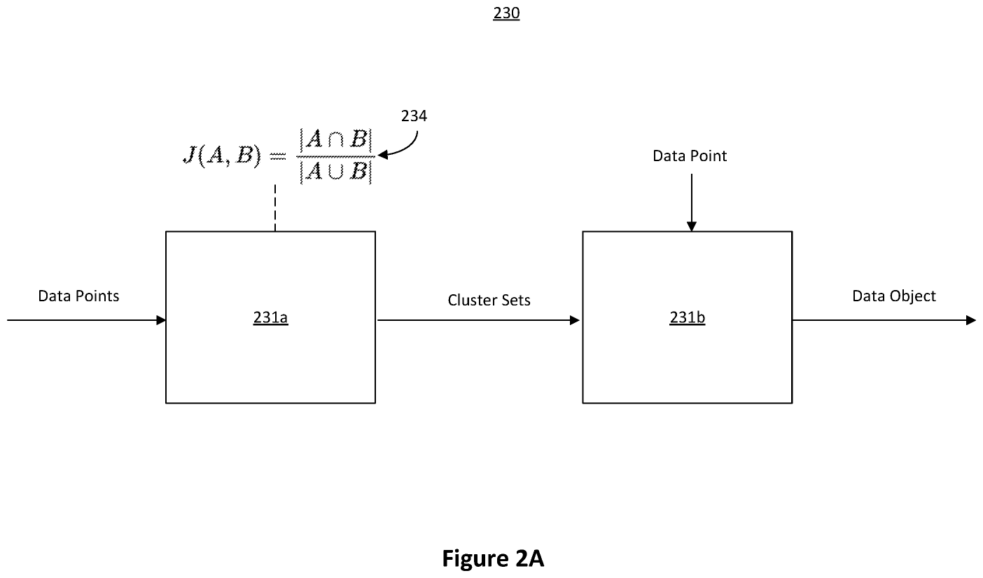

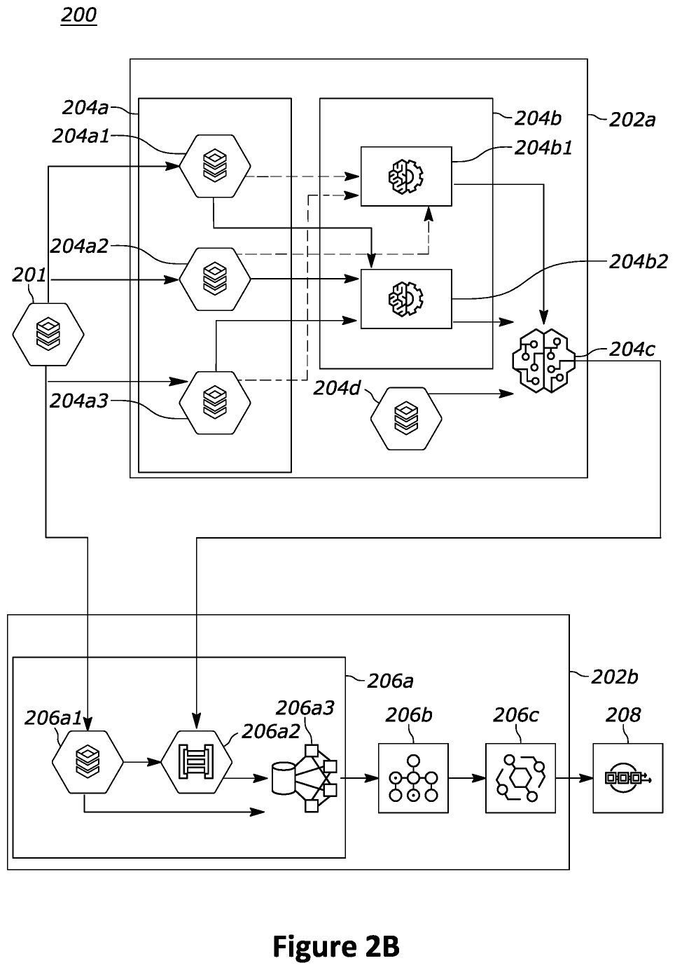

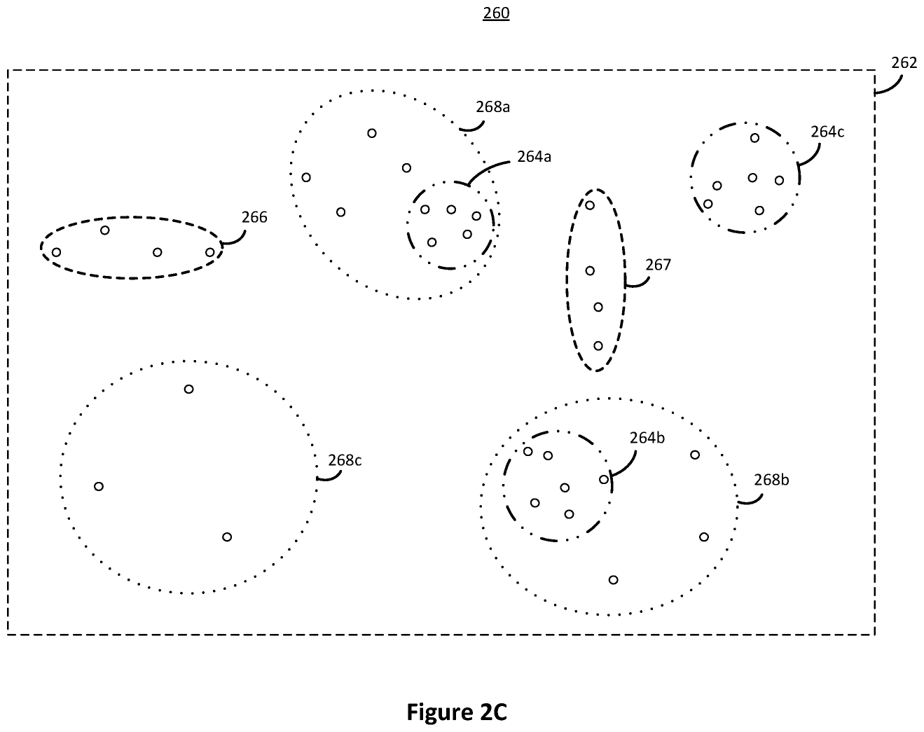

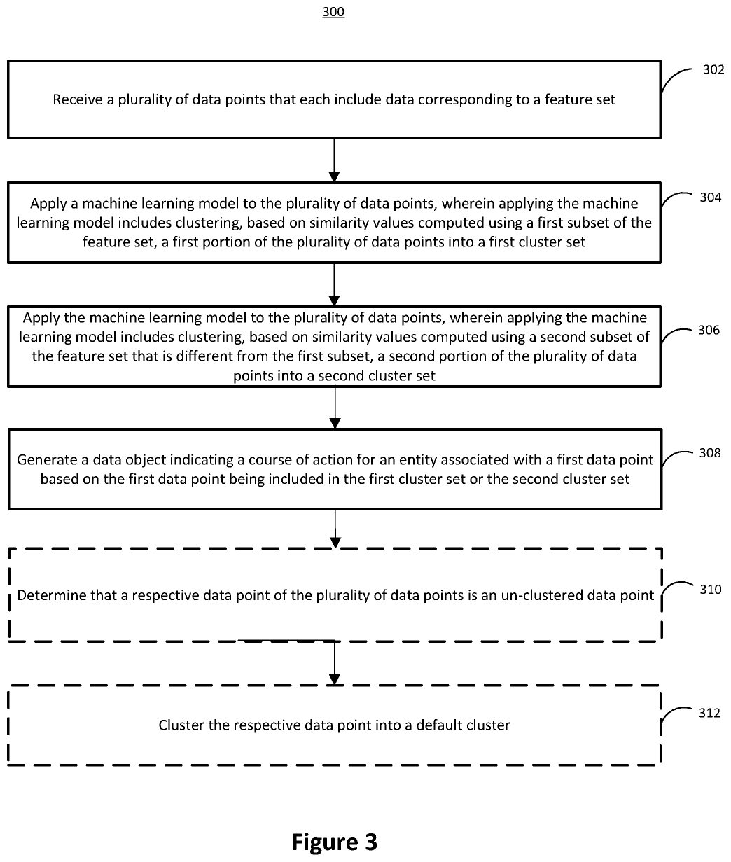

To provide a better understanding of the techniques described herein, FIG. 1 depicts an example computing environment in which techniques of the present disclosure may be implemented, and FIGS. 2A and 2B illustrate how some of these system components may interact and/or otherwise process data to generate clusters, data objects, and/or other output. FIG. 2C depicts an example clustering that the systems/components of FIGS. 2A and 2B may generate. FIG. 3 illustrates an example computer-implemented method for clustering data with a hierarchical clustering model to generate a data object for an entity.

### Example Computing System

FIG. 1 depicts an example computing system 100 in which various embodiments of the present disclosure may be implemented. Depending on the embodiment, the example computing system 100 may generate data clusters, data objects, and/or any related values, responses, or combinations thereof. Of course, it should be appreciated that, while the various components of the example computing system 100 (e.g., central server 102, user device 104, external server 106, etc.) are illustrated in FIG. 1 as single components, the example computing system 100 may include multiple (e.g., dozens, hundreds, thousands) of user devices 104 and external servers 106 that are simultaneously connected to the network 108 at any given time.

Generally, the example computing system 100 includes a central server 102, a user device 104, and an external server 106. The central server 102 receives user data 104b1 from the user device 104 connected to the server 102 through a network 108 and processes the user data 104b1 in accordance with one or more sets of instructions stored in a memory 102b to output any of the values/responses previously described. In certain embodiments, the user data 104b1 is or includes a text string, an audio stream, a video stream, a file, a document, and/or any other suitable data/datatype(s) or combinations thereof. For example, the user data 104b1 may be a patient health chart or medical history documentation indicating current/past illnesses, diagnoses, symptoms, treatments, height, weight, age, gender, and/or other user health data and demographic data for a particular individual. In this example, the central server 102 receives the user data 104b1, executes a machine learning model 102b2 configured to cluster the user data 104b1, and/or generates a data object for the patient based on the cluster(s) output by the machine learning model 102b2.

The central server 102 includes one or more processors 102a, the memory 102b, and a networking interface 102c. The memory 102b stores executable instructions that are configured to, when executed by the one or more processors 102a, cause the one or more processors 102a to analyze data received at the central server 102 and output various values. The machine learning model 102b2, the clustering data 102b3, and the object data 102b4 may all include such executable instructions, as well as other data. The memory 102b may also store additional data and/or databases. It should be appreciated that the central server 102 can include one or multiple computing devices that are co-located or distributed.

The machine learning model 102b2 is generally an artificial intelligence (AI) algorithm/model that utilizes machine learning techniques to perform hierarchical clustering of data points. In certain embodiments, the machine learning model 102b2 is or includes a hierarchical clustering model that evaluates similarity values as a parameter considered at any clustering tier. In some embodiments, the similarity values are Jaccard distances between data points, as described herein. In other embodiments, the machine learning model 102b2 includes and/or utilizes any other suitable machine learning technique, including supervised and/or unsupervised machine learning techniques.

As a general example, a user/operator accessing the central server 102 may submit data associated with medical information, demographic data, and/or other data associated with many anonymized patients to the machine learning model 102b2 for clustering. The machine learning model 102b2 then analyzes the data and creates clusters that each include data associated with two or more patients. In clusters with data corresponding to multiple patients, the data for each patient may share qualitative and/or quantitative similarities with the corresponding data of the other patients represented in the cluster.

In one example, a first cluster includes data associated with multiple, different patients that are each of substantially similar age, similar gender, and have a medical history including a first set of identical/similar co-morbidities. Also in this example, a second cluster includes data that is not included in the first cluster and is associated with multiple, different patients that have correspondingly different medical histories than the patients represented in the first cluster. The machine learning model 102b2 may continue to iteratively perform clustering at an increasing number of clustering tiers until all of the available data is included as part of a cluster and/or until an orphaned data threshold is satisfied. The orphaned data threshold may be a maximum amount of data (e.g., maximum number of data points) included in the available data set that is permitted to remain un-clustered. The orphaned data threshold may be expressed as a percentage/ratio, an integer value, and/or any other suitable quantity.

Continuing the prior example, the machine learning model 102b2 creates the first cluster and the second cluster at the first clustering tier by using a first feature subset. The model 102b2 then proceeds to cluster data that remains un-clustered after the first clustering tier at a second clustering tier by using a second feature subset that is different from the first feature subset. As used throughout this disclosure, references to a “subset” of a feature set may refer to an entirety of the feature set or to less than the entirety of the feature set, with different subsets of a given feature set being either overlapping or non-overlapping. In certain embodiments, for instance, the first feature subset of this example includes every feature of the feature set, and the second feature subset includes fewer features of the feature set. Thus, in this example and embodiment, the machine learning model 102b2 creates clusters at the first clustering tier based on the entire feature set of the available data and creates clusters at the second clustering tier based on less than the entire feature set of the available data.

As noted above, the machine learning model 102b2 is generally configured to utilize artificial intelligence and/or machine learning techniques. These artificial intelligence and/or machine learning techniques include unsupervised machine learning techniques, for example. Machine learning may be implemented through machine learning methods and algorithms. In certain embodiments, the machine learning model 102b2 utilizes a hierarchical clustering model/algorithm to determine meaningful associations between data points and cluster such data points together.

In certain embodiments, the machine learning model 102b2 employs unsupervised learning, which involves finding meaningful relationships in unorganized data. Unlike supervised learning, unsupervised learning does not involve user-initiated training based upon example inputs with associated outputs/labels. Rather, in unsupervised learning, the machine learning model 102b2 organizes unlabeled data according to a relationship determined by at least one machine learning method/algorithm employed by the machine learning model 102b2. Unorganized data may include any combination of data inputs and/or machine learning outputs, as described above.

In some embodiments, at least one of a plurality of machine learning methods and algorithms are applied, which may include but are not limited to: cluster analysis, k-nearest neighbor algorithms, and/or other ML programs/algorithms either individually or in combination.

In various embodiments, the implemented machine learning methods and algorithms are directed toward at least one of a plurality of categorizations of machine learning, such as unsupervised learning.

It is to be understood that unsupervised machine learning may also comprise retraining, relearning, or otherwise updating models with new, or different, information, which may include information received, ingested, generated, or otherwise used over time. Further, it should be appreciated that, as previously mentioned, the machine learning model 102b2 may be used to output a cluster, cluster set, data object (e.g., care path), and/or any other values, responses, or combinations thereof using artificial intelligence (e.g., a machine learning model of the machine learning model 102b2) or, in alternative aspects, without using artificial intelligence.

Moreover, although the methods described elsewhere herein may not directly mention machine learning techniques, such methods may be read to include such machine learning for any determination or processing of data that may be accomplished using such techniques. Such machine learning models/algorithms may, therefore, be used to perform part or all of the analytical functions of the methods described elsewhere herein. In some aspects, such machine learning techniques may be implemented automatically upon occurrence of certain events or upon certain conditions being met. In any event, use of machine learning techniques, as described herein, may begin with training a machine learning program, or such techniques may begin with a previously trained machine learning program.

The clustering data 102b3 generally facilitates these clustering actions performed by the machine learning model 102b2. In particular, the clustering data 102b3 includes instructions that, when executed by the one or more processor(s) 102a cause the processors 102a to apply the machine learning model 102b2 to any received input data. Further, the clustering data 102b3 also includes the data received at the central server 102 from, for example, the user device 104 (e.g., user data 104b1).

In certain embodiments, the clustering data 102b3 includes feature sets and/or parameter sets that are applied at a clustering tier of the machine learning model 102b2. As an example, at a first clustering tier, the processors 102a retrieves/accesses data from the clustering data 102b3 indicating which features the machine learning model 102b2 should evaluate. In this example, the data retrieved/accessed from the clustering data 102b3 causes the machine learning model 102b2 to evaluate a first feature subset that includes the entire feature space. Thus, in this example, the machine learning model 102b2 analyzes/compares data point(s) for each available feature of the data to develop a first cluster set. Continuing this example, and at a second clustering tier, the clustering data 102b3 indicates that the machine learning model 102b2 should evaluate a second feature subset that consists of less than the entire feature space for the available data, to potentially cluster data that remained un-clustered after the first clustering tier.

As a practical example, the feature space for a data set evaluated by the machine learning model 102b2 may include a co-morbidity value, a co-morbidity severity value, an age value, a gender value, a procedure code, a diagnosis code, and a service location. At a first clustering tier, the clustering data 102b3 indicates that the machine learning model 102b2 should cluster available data based on the similarities of data points associated with all available features in the feature set. However, it may be known a priori and/or otherwise determined by the machine learning model 102b2 that clustering based on the co-morbidity severity value feature produces a substantial amount of orphaned data. Thus, at a second clustering tier, the clustering data 102b3 indicates that the machine learning model 102b2 should cluster available data based on the similarities of data points associated with all features except the co-morbidity severity value. Advantageously, and as previously mentioned, this multi-tiered clustering based on flexible and/or customizable feature sets achieves lower orphaned data rates than conventional techniques, which yields more clusters from which to draw/develop insights (e.g., identify or select care paths), and thereby increases the overall utility of data received at the central server 102.

The object data 102b4 broadly includes instructions and/or data that, when executed, causes the one or more processors 102a to generate a data object indicating a course of action for an entity based on the clustered data output by the machine learning model 102b2. In certain embodiments, the data object is or includes a care path and the course of action indicated by the care path is a recommended course of treatment and/or actions that a patient may take, and/or a healthcare provider may take with respect to the patient, to reduce symptoms, mitigate pain, cure an illness, and/or otherwise improve or maintain the patient's health. Such treatment and/or actions include, for example and without limitation, medications, physical/mental exercises/activities, diets or eating plans, and/or medical procedures (e.g., surgery). Of course, the data object and indicated course of action may include any suitable data.

In certain embodiments, generating the data object includes the one or more processors 102a retrieving/accessing data/instructions from the object data 102b4 to identify a care path associated with a cluster that most closely aligns with the symptoms and/or other information of the patient, and returning an indication of the care path (e.g., text description, and/or images, tutorial video, etc.) for viewing by the patient. However, in some embodiments, generating the data object includes the central server 102 causing the user device 104 to display an indication of a cluster the user/patient was clustered with following the execution of the machine learning model 102b2.

Further, in instances where no care path is available, the processor(s) 102a may generate a data object by generating a new care path for the patient. In these instances, the object data 102b4 may instruct the one or more processors 102a to, for example, determine care paths that are associated with clusters proximate and/or otherwise similar to the patient's cluster and generate a new care path by aggregating and adjusting the recommended actions of such similar care paths. These new care paths may thereby include recommended actions that are estimated to provide optimal care for the patient without requiring predetermined care paths for every possible cluster and/or combination of data points.

In some embodiments, the machine learning model 102b2 and/or any other suitable component(s) output such data objects by receiving inputs from the object data 102b4 and based on similarities between the patient's expressed symptoms, medical history, demographic data, and/or other data, and the data included in clusters output by the machine learning model 102b2. Additionally, or alternatively, the object data 102b4 may include all instructions necessary to generate the care path without requiring the machine learning model 102b2.

Generally, the user device 104 is or includes any device that is associated with (e.g., owned and/or operated by) a particular user/patient, who may provide data that is transmitted to the central server 102 and/or the external server 106 through the network 108. In certain embodiments, the user device 104 is a personal computing device of that user, such as a smartphone, a tablet, smart glasses, or any other suitable device or combination of devices (e.g., a smart watch plus a smartphone) with wireless communication capability. In the embodiment of FIG. 1, the user device 104 includes a processor 104a, a memory 104b, a networking interface 104c, and a display 104d. The memory 104b stores user data 104b1.

In some embodiments, the user data 104b1 includes medical information (e.g., co-morbidities, symptoms, age, gender, other demographic data, etc.) for the user/patient associated with user device 104. The central server 102 may receive an initial communication from the user device 104 when establishing a communication stream across a communication channel, for example, and this initial communication may include the user data 104b1. The central server 102 may then access/retrieve the locally-stored user data 104b1 to cluster the data 104b1, generate a data object based on the clustering of the data 104b1, cause the device 104 to display information for the user/patient (e.g., via display 104d), and/or otherwise communicate with the user device 104.

For example, data stored as part of the user data 104b1 corresponding to a patient may indicate that the patient is a 53 year old female with obesity, diabetes, and high blood pressure as co-morbidities, a relatively high co-morbidity severity value for these co-morbidities based on the patient's demographic data, diagnosis codes corresponding to diabetes and high blood pressure, and a service location indicating treatment for the co-morbidities at a healthcare location. In this example, the central server 102 receives an initial communication across the network 108 from the user device 104 associated with the patient, and the central server 102 executes the machine learning model 102b2 to cluster the patient's data with a set of pre-existing data clusters. Once clustered, the one or more processors 102a execute instructions included as part of the object data 102b4 to generate a data object for the patient. Thus, the central server 102 may utilize the user data 104b1 to improve the known data clusters by incorporating additional patient data and may generate relevant data object(s) for the first patient that correspond to the clustering of the first patient's data. Alternatively, the machine learning model 102b2 re-clusters all patient data included in the pre-existing data clusters with the patient's data to create new clusters.

The user device 104 is communicatively coupled to the central server 102 and/or the external server 106. For example, the user device 104, the central server 102, and/or the external server 106 may communicate via USB, Bluetooth, Wi-Fi Direct, Near Field Communication (NFC), etc. For example, the central server 102 may transmit a cluster indication, a data object indication, and/or any other values, responses, or combinations thereof to the user device 104 via the networking interface 102c, which the user device 104 may receive via the networking interface 104c.

The external server 106 may be or include computing servers and/or combinations of multiple servers storing data that may be accessed/retrieved by the central server 102 and/or the user device 104. In certain embodiments, the external server 106 receives data from the central server 102 and/or the user device 104 and retrieves/accesses information stored in memory 106b for transmission back to the central server 102 and/or the user device 104. The external server 106 may include a processor 106a, a memory 106b, and a networking interface 106c. It should be appreciated that the external server 106 can include one or multiple computing devices that are co-located or distributed.

Further, in certain embodiments, the external server 106 includes data from one or both of the user device 104 and/or the central server 102. In one such example, the external server 106 is a server located in and/or otherwise associated with a hospital or other healthcare provider, and the external server 106 includes electronic health records in memory 106b. These electronic health records may be or include the user data 104b1. As another example, the external server 106 serves as a database for some/all of the clustering data 102b3 and/or some/all of the object data 102b4. In some embodiments, the example computing system 100 does not include the external server 106.

Each of the processors 102a, 104a, 106a may include any suitable number of processors and/or processor types. For example, the processors 102a, 104a, 106a may each include one or more CPUs and one or more graphics processing units (GPUs). Generally, each of the processors 102a, 104a, 106a may be configured to execute software instructions stored in each of the corresponding memories 102b, 104b, 106b. The memories 102b, 104b, 106b may each include one or more persistent memories (e.g., a hard drive and/or solid state memory) and may store one or more applications, modules, and/or models, such as the machine learning model 102b2.

The networking interface 102c may enable the central server 102 to communicate with the user device 104, the external server 106, and/or any other suitable devices or combinations thereof. More specifically, the networking interface 102c enables the central server 102 to communicate with each component of the example computing system 100 across the network 108 through their respective networking interfaces 104c, 106c. The networking interfaces 102c, 104c, 106c may support wired or wireless communications, such as USB, Bluetooth, Wi-Fi Direct, Near Field Communication (NFC), etc. The networking interface 102c may enable the central server 102 to communicate with the various components of the example computing system 100 via a wireless communication network such as a fifth-, fourth-, or third-generation cellular network (5G, 4G, or 3G, respectively), a Wi-Fi network (802.11 standards), a WiMAX network, or any other suitable wide area network (WAN), local area network (LAN), or personal area network (PAN), etc.

Moreover, the network 108 may be a single communication network, or may include multiple communication networks of one or more types (e.g., one or more wired and/or PANs or LANs, and/or one or more WANs such as the Internet). In some embodiments, the network 108 includes multiple, entirely distinct networks (e.g., one or more networks for communications between central server 102 and user device 104, and a separate, Bluetooth or wireless LAN (WLAN) network for communications between central server 102 and user device 104, and so on).

It will be understood that the above disclosure is one example and does not necessarily describe every possible embodiment. As such, it will be further understood that alternate embodiments may include fewer, alternate, and/or additional steps or elements.

### Example Cluster-Based Data Object Generation Architecture and Sequences

FIG. 2A depicts an example data point clustering and data object generation sequence 230, in accordance with various embodiments described herein. The example data point clustering and data object generation sequence 230 broadly illustrates a clustering stage 231a and a data object generation stage 231b, which may be performed by central server 102 (e.g., processor 102a and/or other components of central server 102) of FIG. 1, for example. The data point clustering and data object generation sequence 230 illustrated in FIG. 2A is for the purposes of discussion only, and additional/alternative data point clustering and/or data object generation sequences utilizing additional/alternative machine learning techniques may also, or instead, be utilized.

Initially, the clustering stage 231a receives a set of data points, as described herein. The data points may include feature data (e.g., corresponding to geographic data, medical condition data, age bracket data, etc.) for an individual and/or a group of individuals. As one example, the input data points may represent a set of data corresponding to a particular patient who is requesting an optimal care path to treat a medical condition. As another example, the input data points may include data from a plurality of patients used to create cluster sets by leveraging a machine learning model (e.g., machine learning model 102b2). Regardless, the clustering stage 231a includes utilizing the input data points to determine cluster sets, which also includes determining a particular cluster set in which the data points for a particular individual are clustered.

Determining a cluster set also utilizes the Jaccard distance metric 234, as described herein (e.g., with respect to FIG. 2B). Specifically, the clustering stage 231a includes analyzing the input data points by determining at least a Jaccard distance 234 between some/all of the sets of data points and clustering the data points together into clusters based, at least in part, on the Jaccard distance 234. In certain embodiments, the clustering stage 231a includes analyzing any suitable set of features included in the data points to calculate the Jaccard distance 234, and any suitable set of parameters to determine whether a particular data point should be included in a cluster and/or whether a particular cluster is suitable for creation/storage.

More specifically, certain potential clusters may not satisfy each of the parameters at a particular clustering tier, and as a result may not include data points of sufficient similarity to generate reliable data objects. Thus, the clustering stage 231a may not include creating/generating these potential clusters for storage as part of the cluster sets. For example, a minimum cluster size to create a cluster may be ten distinct data points and a potential cluster created at the clustering stage 231a may include five distinct data points. If no further distinct data points are added to the potential cluster by the end of the current tier of hierarchical clustering, the potential cluster may be dissolved, rejected, and/or not saved as part of the cluster set from the current tier of hierarchical clustering. Thus, in this example, each of the distinct data points included as part of the potential cluster may remain un-clustered at the end of the current tier of hierarchical clustering.

The data object generation stage 231b includes generating a data object (e.g., as discussed in FIG. 2B and elsewhere herein) to generate a data object based on the cluster sets. Namely, the data object generation stage 231b includes receiving a data point corresponding to a particular patient/individual requesting a data object (e.g., care path), which includes data associated with the particular patient/individual corresponding to a feature set (e.g., geographic data, age brackets data, medical conditions data). In certain embodiments, the data point received at the data object generation stage 231b is, or is included as part of, the data points received at the clustering stage 231a.

Further, the data object generation stage 231b includes executing a machine learning model (e.g., machine learning model 102b2) to cluster and/or otherwise determine a cluster in which the data point is included. The data object generation stage 231b then includes determining a data object for recommendation to the particular patient/individual based on known and/or predicted/generated data objects associated with the cluster. For example, a first cluster may be associated with a first condition and a first age bracket, and may correspondingly be associated with a first care path with a first set of treatments, actions, prognoses, risks, and/or other data intended to cure, mitigate, and/or otherwise treat the first condition and/or associated co-morbidities. In this example, a second cluster may be associated with a second condition and a second age bracket that are different from the first condition and the first age bracket and may correspondingly be associated with a second care path with a second set of treatments, actions, prognoses, risks, and/or other data that is different from those included in the first care path.

In certain embodiments, a central server (e.g., central server 102) initiates/causes performance of the data object generation represented by the data object generation stage 231b. The central server 102 may initiate/cause performance of the data object generation by, for example, generating a display of the data object on an output device or transmitting a message that triggers/causes another device (e.g., user device 104) to display the data object for a patient/user. In other words, the central server 102 can initiate/cause performance of the data object generation by causing any suitable device to display a generated data object, such as a care path output as a result of the data object generation stage 231b.

FIG. 2B depicts an example cluster-based data object generation architecture 200, in accordance with some of the embodiments described herein. Generally, the example architecture 200 includes a symmetry component 201, a segmentation branch 202a, and a data object branch 202b. The symmetry component 201 is configured to transmit data to the segmentation branch or the data object branch 202b. In certain embodiments, the symmetry component 201 transmits updated/new feature set data (e.g., medical data, demographic data, geographic data, etc.) to the segmentation branch 202a and updated/new data object data (e.g., recommended treatment chronologies, etc.) to the data object branch 202b. The segmentation branch 202a is configured to receive input user/patient data and output data clusters that include different portions of the input user/patient data. The data object branch 202b is configured to receive the data clusters from the segmentation branch 202a and an individual patient's data (and/or a group of patients' data) to output a data object for each patient represented in the input data. In certain embodiments, each of the actions described herein in reference to the example cluster-based data object generation architecture 200 are performed by a central server (e.g., central server 102).

The segmentation branch 202a generally includes a member data section 204a, an initial segmentation section 204b, and a clustering algorithm 204c. Initially, the segmentation branch 202a receives information/data from the symmetry component 201 to update and/or otherwise refresh the data included in the member data section 204a. The member data section 204a generally includes feature set data, such as geographic data 204a1, medical conditions data 204a2, and age brackets data 204a3.

The geographic data 204al generally includes geographic data/information, such as service locations, associated with entities/individuals requesting a data object (e.g., care path) and/or otherwise included in the clustering functions described herein. For example, service location corresponding to a first patient may indicate that the first patient received a particular healthcare service (e.g., vaccination, surgery, physical examination, etc.) in a region of a country, a particular county/state/principality, a city/town, and/or any other indication of the service location at any suitable granularity. Additionally, the geographic data 204al may include other geographic data corresponding to individuals/entities requesting a data object and/or otherwise included in the clustering functions described herein.

The medical conditions data 204a2 generally includes data corresponding to medical conditions individuals/entities requesting a data object (e.g., care path) and/or otherwise included in the clustering functions described herein experienced and/or are currently experiencing. For example, medical condition data corresponding to a second patient may indicate that the second patient experienced a particular co-morbidity symptom/condition that has a corresponding co-morbidity severity value, and the medical condition data may further indicate a diagnosis code indicating a formal diagnosis of that co-morbidity symptom/condition. Additionally in this example, the medical conditions data 204a2 may include a procedure code value/data corresponding to a procedure that the second patient received and/or may receive to alleviate and/or otherwise address the co-morbidity symptom/condition. Additionally, the medical conditions data 204a2 may include other data corresponding to individuals/entities requesting a data object and/or otherwise included in the clustering functions described herein, such as condition data, prognosis data, health record data, and/or other suitable data or combinations thereof.

The age brackets data 204a3 generally includes demographic data corresponding to individuals/entities requesting a data object (e.g., care path) and/or otherwise included in the clustering functions described herein. For example, age brackets data corresponding to a third patient may indicate that the third patient is within a particular age bracket including patients from ages 35 to 45 and the third patient is a biological male. Additionally, the age brackets data 204a3 may include other demographic data corresponding to individuals/entities requesting a data object and/or otherwise included in the clustering functions described herein, such as height data, weight data, ethnicity data, and/or other suitable data or combinations thereof.

As mentioned, the segmentation branch 202a also includes an initial segmentation section 204b. The initial segmentation section 204b generally includes a Bernoulli mixture model 204b1 and Jaccard clusters 204b2. Broadly, the Bernoulli mixture model 204b1 is a statistical model that can combine multiple Bernoulli distributions to describe complex data, such as the data included in the member data section 204a. The Bernoulli mixture model 204b1 generally receives each of the geographic data 204al, the medical conditions data 204a2, and the age brackets data 204a3, and outputs distributions and/or distribution models corresponding to some/all of the data contained therein.

Namely, each of the data included as part of the geographic data 204al, the medical conditions data 204a2, and the age brackets data 204a3 may be represented as binary data, and each of these data may be considered independent variables. These variables may then follow individual Bernoulli distributions, which capture the joint behavior of such binary variables. The Bernoulli mixture model 204b1 may generate and/or analyze these individual Bernoulli distributions to further generate a combined Bernoulli distribution/model representing the geographic data 204a1, the medical conditions data 204a2, and the age brackets data 204a3 for each individual/entity. In doing so, the Bernoulli mixture model 204b1 captures intricate patterns, dependencies, correlations, and/or other relationships existing between/among the geographic data 204a1, the medical conditions data 204a2, and/or the age brackets data 204a3 for each individual/entity and/or groups of individuals/entities.

The Jaccard clusters 204b2 generally represent clusters of the data included in the member data section 204a. Namely, the Jaccard clusters 204b2 include such clusters of data based on the Jaccard distance metric, given by:

$$J ( A , B ) = \frac{\vert A ⋂ B \vert}{\vert A ⋃ B \vert} , \qquad ( 1 )$$

where J is the Jaccard distance, A is a first data set from the member data section 204a, and B is a second data set form the member data section 204a.

For example, a first data set A may include geographic data 204al, medical conditions data 204a2, and/or age brackets data 204a3 corresponding to a first patient who is a 53-year-old female that sought treatment for a first condition and a first co-morbidity at a service location in a first region. Further in this example, and a second data set B may include geographic data 204a1, medical conditions data 204a2, and/or age brackets data 204a3 corresponding to a second patient who is a 22-year-old male that sought treatment for a second condition at a service location in a second region. Thus, in this example, the first patient is in a different age bracket than the second patient, sought treatment for a different condition and co-morbidity than the second patient, and received treatment in a different service location than the second patient. Accordingly, the Jaccard distance, given by equation (1), between the first data set A and the second data set B may be relatively small (e.g., <0.25), as the similarities between the first data set A and the second data set B are relatively small.

In a second example, a first data set C may include geographic data 204al, medical conditions data 204a2, and/or age brackets data 204a3 corresponding to a first patient who is a 72-year-old male that sought treatment for a first condition and a first co-morbidity at a service location in a first region. Further in this example, and a second data set D may include geographic data 204al, medical conditions data 204a2, and/or age brackets data 204a3 corresponding to a second patient who is a 74-year-old male that sought treatment for the first condition at a service location in the first region. Thus, in this example, the first patient is in the same age bracket (e.g., 70 to 80) as the second patient, sought treatment for an identical condition but a different co-morbidity than the second patient, and received treatment in an identical service location as the second patient. Accordingly, the Jaccard distance, given by equation (1), between the first data set C and the second data set D may accordingly be relatively large (e.g., >0.75), as the similarities between the first data set C and the second data set D are relatively large.

Regardless, the Jaccard clusters 204b2 may be provided to the clustering algorithm 204c for further processing, as described herein. Namely, the clustering algorithm 204c receives the Bernoulli distributions/models and Jaccard clusters 204b2 from the initial segmentation section 204b and the propensity scores 204d. In certain embodiments, the clustering algorithm 204c is and/or includes the machine learning model 102b2, or a portion thereof. Accordingly, the clustering algorithm 204c is configured to output clusters of data points that are similar, based on the data received from the initial segmentation section 204b and the propensity scores 204d. The propensity scores 204d generally include data input to the clustering algorithm 204c to provide contextual interpretations of various data input to the clustering algorithm 204c from the initial segmentation section 204b.

In any event, when the clustering algorithm 204c receives the inputs from the initial segmentation section 204b and the propensity scores 204d, the algorithm 204c performs a hierarchical clustering to cluster the input data into cluster sets. The hierarchical clustering generally includes the clustering algorithm 204c clustering data points from the member data section 204a into cluster sets based on different feature subsets from the data points at different tiers of the clustering hierarchy. This hierarchical clustering further includes potentially applying different subsets of a parameter set at the different tiers of the clustering hierarchy.

For example, in a first tier of the hierarchical clustering of the clustering algorithm 204c, the algorithm 204c may cluster a first set of data points into a first cluster set based on similarities (e.g., based on the Jaccard distance metric) of each of the geographic data 204al, the medical conditions data 204a2, and the age brackets data 204a3 for each data point of the first set of data points. The first tier of hierarchical clustering may also include a parameter set including a data point neighbor value, a minimum cluster size, and a distance threshold (e.g., Jaccard distance).

The parameter set at any given tier of the hierarchical clustering performed by the clustering algorithm 204c generally includes parameters for determining which data points to include in a cluster set. For example, the data point neighbor value generally corresponds to how many neighboring data points the clustering algorithm 204c must locate for an individual data point to include the individual data point in a data cluster. In other words, for any data point, the clustering algorithm 204c may attempt to identify the threshold number of other proximate data points (i.e., “neighboring” data points). If the algorithm 204c is successful in identifying the threshold number of neighboring data points, the algorithm 204c may stop evaluating neighboring data points for that particular data point and move on to other data points. The minimum cluster size may define the minimum number of data points that are allowed to substantiate a cluster. Namely, if the clustering algorithm 204c determines that a first number of data points are otherwise suitable to cluster together into a cluster, but that the first number is less than the minimum cluster size, the algorithm 204c will not cluster the data points into a cluster. Moreover, the distance threshold may define the maximum Jaccard distance between two data points for the two data points to be considered/clustered together in the same cluster. If, for example, the distance threshold is set to 1, then the clustering algorithm 204c will only cluster data points together into clusters that are exact matches (e.g., data/values of the considered feature set are identical).

Regardless, continuing the prior example, a second set of data points remain un-clustered after the first tier of clustering performed by the clustering algorithm 204c. In a second tier of the hierarchical clustering of the clustering algorithm 204c, the algorithm 204c may cluster the second set of data points into a second cluster set based on similarities (e.g., based on the Jaccard distance metric) of the geographic data 204al and the medical conditions data 204a2 for each data point of the second set of data points. Additionally, the clustering algorithm 204c may only consider the Jaccard distance metric threshold and a minimum cluster size threshold as parameters enforced against clustering of data points at the second tier of hierarchical clustering.

Thus, in this second tier of hierarchical clustering, the algorithm 204c does not consider similarities between the age brackets data 204a3 of the data points in the second set of data points and/or the data point neighbor value parameter. In this manner, the clustering algorithm 204c may potentially recognize sets of data points that have substantial similarities between/among certain subsets of the entire feature space (e.g., data in the member data section 204a) that otherwise fail to satisfy the parametric considerations/thresholds enforced at the first tier of hierarchical clustering.

Still further in this example, there is a third set of data points that remain un-clustered after the first and second tiers of clustering performed by the clustering algorithm 204c. In a third tier of the hierarchical clustering of the clustering algorithm 204c, the algorithm 204c may cluster the third set of data points into a third cluster set based on similarities (e.g., based on the Jaccard distance metric) of the medical conditions data 204a2 for each data point of the second set of data points. Additionally, the clustering algorithm 204c may only consider the Jaccard distance metric threshold as a parameter enforced against clustering of data points at the third tier of hierarchical clustering.

Thus, in this third tier of hierarchical clustering, the algorithm 204c does not consider similarities between the geographic data 204al or the age brackets data 204a3 of the data points in the third set of data points and/or the data point neighbor value or minimum cluster size parameters. In this manner, the clustering algorithm 204c may potentially recognize sets of data points that have substantial similarities between/among certain subsets of the entire feature space (e.g., data in the member data section 204a) that otherwise fail to satisfy the parametric considerations/thresholds enforced at the first tier and the second tier of hierarchical clustering.

When the clustering algorithm 204c generates/outputs the various cluster sets, the clustering algorithm 204c transmits the cluster sets to the data object branch 202b. In particular, the data object branch 202b receives the cluster sets from the clustering algorithm 204c and data from the symmetry component 201. This data may include various carepaths 206al that each generally indicate a series of treatments, therapies, procedures, and/or other actions configured to alleviate, mitigate, cure, and/or otherwise impact the symptoms of the various conditions and/or co-morbidities indicated in the cluster sets. To link these carepaths 206al to the cluster sets, the data object branch 202b includes a graph data creation section 206a that includes generating, creating, and/or otherwise constructing a transition matrix 206a2.

Broadly, the transition matrix 206a2 combines the carepaths 206al and cluster sets in a configuration that represents the probabilistic association between an individual cluster set and an individual carepath. These probabilistic associations are represented as connections between nodes of the carepath graph 206a3. In other words, each entry within the transition matrix 206a2 indicates whether a connection between two corresponding nodes of the carepath graph 206a3 exists and the probability of moving from a vertex to another in a single step. This combination of the transition matrix 206a2 and the carepath graph 206a3 thereby generally represent the probabilistic optimal carepath(s) for an individual that is clustered into any known cluster determined by the clustering algorithm 204c.

The processors of the systems described herein (e.g., processors 102a, processors 106a) also analyze the carepath graph 206a3 to generate node embeddings 206b for the nodes of the carepath graph 206a3. Generally, node embedding is a technique used to represent nodes in a graph as low-dimensional vectors. These embeddings thereby capture the structural information of the graph represented by the nodes in a compact and meaningful manner. In certain embodiments, the processors of the present techniques (e.g., processors 102a, processors 106a) train a neural network or other machine learning model/algorithm to predict the context of each node in the graph output a low-dimensional vector (i.e., embedding) that represents each node. As a result, the node embeddings 206b may enable processors of the present techniques to predict whether two nodes are connected by an edge.

The processors of the present techniques (e.g., processors 102a, processors 106a) may take these embedded nodes and perform segment clustering 206c with such nodes. Generally speaking, segment clustering is used to uncover patterns and structures within complex networks/graphs. In certain embodiments, the segment clustering 206c performed by the processors of the present techniques is and/or utilizes an unsupervised learning model/technique (e.g., hierarchical clustering) that involves partitioning the embedded nodes of the carepath graph 206a3 into clusters based on certain common/shared characteristics. Nodes assigned to the same cluster may share more properties with each other than with nodes in different clusters.

With the clustered nodes output as a result of the segment clustering 206c, the data object branch 202b may output a carepath 208. As previously mentioned, a carepath 208 generally includes a series of treatments, therapies, procedures, and/or other actions configured to alleviate, mitigate, cure, and/or otherwise impact the symptoms of the various conditions and/or co-morbidities indicated in the cluster sets. In certain embodiments, the carepath 208 also indicates a prognosis value, future co-morbidities, future conditions, and/or other circumstance a patient may experience based on the patient's cluster. However, it should be appreciated that the carepath 208 may include any suitable values or data described herein, and/or combinations thereof.

Practically speaking, the carepath 208 output by the data object branch 202b may include a set of classification values and/or classifications that have associated confidence value(s)/interval(s). For example, the carepath 208 may include a first predicted carepath with a confidence value of 80%. Of course, the carepath 208 may be or include predicted actions, treatments, etc. with confidence values indicated in any suitable manner, such as a single numerical value (e.g., 1, 2, 3, etc.), a confidence interval, a percentage (e.g., 95%, 50%, etc.), an alphanumerical character(s) (e.g., A, B, C, etc.), a symbol, and/or any other suitable value or indication of a likelihood that the carepath 208 is accurate.

In some embodiments, the data object branch 202b outputs the carepath 208 as a set or list of classification values corresponding to the likely carepath(s) for a particular patient(s). For example, the data object branch 202b may output a first carepath with a reference/indication to a first cluster with a confidence value of 95%, a second carepath with a reference/indication to a second cluster with a confidence value of 75%, and a third carepath with a reference/indication to a third cluster with a confidence value of 45%.

FIG. 2C depicts an example data point clustering 260 using different feature sets, in accordance with various embodiments described herein. Broadly, the example data point clustering 260 includes data points clustered into multiple cluster sets within a feature space graph 262. The cluster sets 264a-c, 266, 267, and 268a-c represent data points that the systems described herein have clustered together based on subsets of a feature set and subsets of a parameter set.

Practically speaking, the feature space may be an N-dimensional space, where N is any integer and represents the total number of features associated with any data point. In such an N-dimensional feature space, each data point may be positioned based on an N-dimensional vector with values associated with the feature values for that data point. For example, each data point may include feature values associated with a co-morbidity value, a co-morbidity severity value, an age value, a gender value, a procedure code, a diagnosis code, a service location, and/or any other suitable value(s) or combinations thereof. The systems described herein may evaluate these vectors and/or the component values to determine the clusters.

However, for simplicity, the feature space represented by the feature space graph 262 in FIG. 2C is a two-dimensional space, such that the data points included therein have two corresponding features. The position of the data points within the feature space graph 262 correspond to the values of the features of those data points, such that the relative similarities/differences in positions of two data points reflects the corresponding similarities/differences of the values of those two data points. The cluster sets 264a-c, 266, 267, and 268a-c may generally reflect clustering based on different subsets of the feature set and/or subsets of the parameter set utilized by the systems described herein.

The systems described herein may generate the first cluster set 264a-c during a first clustering tier, wherein data points are evaluated based on all features of the feature space in relation to the parameter set. In one such example, the parameters of interest during the first clustering tier include a distance threshold (e.g., Jaccard distance metric of equation (1)), a minimum cluster size, and a data point neighbor value, as applied to all features of the feature space, which includes a co-morbidity value and a patient's age. During this first clustering tier, the systems described herein (e.g., machine learning model 102b2) may evaluate the feature values of each data point within the first cluster 264a and determine that each data point satisfies the distance threshold relative to every other data point and the data point neighbor value, and the aggregate number of data points satisfies the minimum cluster size threshold. In other words, the data points included in the first cluster 264a represent entities (i.e., patients) who are similar/identical ages and have similar/identical co-morbidities. Similarly, the systems described herein may evaluate all the feature values of the data points in the second cluster 264b and the third cluster 264c, in accordance with the parameters of interest, to determine whether each data point included therein should be included as part of the respective clusters. Thus, the first cluster set 264a-c includes three clusters 264a, 264b, and 264c, each including data points that satisfy the parametric constraints/thresholds associated with all features of the feature space. Of course, in certain embodiments, the cluster sets described herein may include any suitable number of clusters.

As an example, the systems described herein may generate the second cluster set 266 (represented in FIG. 2C as a single cluster) during a second clustering tier, wherein data points are evaluated based on fewer than all features of the feature space in relation to the parameter set. In this example, the feature of interest during the second clustering tier is the co-morbidity value, and the parameters of interest include the distance threshold, the minimum cluster size, and the data point neighbor value, as applied to the co-morbidity value. During this second clustering tier, the systems described herein may evaluate the co-morbidity values of each data point within the second cluster set 266 and determine that each data point therein satisfies the distance threshold and the data point neighbor value, based on the co-morbidity value of the other data points, and the aggregate number of data points satisfies the minimum cluster size threshold. In other words, the data points included in the second cluster set 266 represent entities (i.e., patients) who have similar/identical co-morbidities, but do not have substantially similar ages.

In another example, the feature of interest during the third clustering tier is the age value, and the parameters of interest include the distance threshold, the minimum cluster size, and the data point neighbor value, as applied to the age value. During this third clustering tier, the systems described herein may evaluate the age values of each data point within the third cluster set 267 (represented in FIG. 2C as a single cluster) and determine that each data point therein satisfies the distance threshold and the data point neighbor value, based on the age value of the other data points, and the aggregate number of data points satisfies the minimum cluster size threshold. In other words, the data points included in the third cluster set 267 represent entities (i.e., patients) who have similar/identical age values, but do not have substantially similar co-morbidities.

In certain embodiments, the systems described herein create clusters that include smaller sub-clusters that were clustered as part of a prior clustering tier, and/or may relax or exclude parameters from consideration during certain clustering tiers. In one such example, during a fourth clustering tier, the features of interest include some or all features of the feature space, and the parameters of interest include an adjusted distance threshold, a reduced minimum cluster size, and an adjusted data point neighbor value, as applied to the feature values. The adjusted distance threshold may be an increased/decreased distance threshold that allows data points that failed to satisfy the distance threshold during prior clustering tiers (i.e., Jaccard distance was too low) to potentially satisfy the distance threshold during the fourth clustering tier. Similarly, the reduced minimum cluster size is a smaller minimum cluster size than was required in prior clustering tiers (e.g., 5 data points to 3 for a cluster) to allow data points that did not have sufficient proximate data points to comprise a cluster during prior clustering tiers to potentially satisfy the minimum cluster size during the fourth clustering tier. The adjusted data point neighbor value may be an increased/decreased data point neighbor value that allows data points that failed to satisfy the data point neighbor value during prior clustering tiers (i.e., insufficient neighboring data points) to potentially satisfy the data point neighbor value during the fourth clustering tier.

In any event, during this fourth clustering tier, the systems described herein may evaluate the feature values of each data point within the fourth cluster set 268a-c and determine that each data point therein satisfies the adjusted distance threshold and the adjusted data point neighbor value, based on the feature values of the other data points, and the aggregate number of data points satisfies the reduced minimum cluster size threshold. In other words, the data points included in the first cluster 268a, the second cluster 268b, and the third cluster 268c represent entities (i.e., patients) who have similar/identical age values and/or have similar/identical co-morbidities. In particular, the first cluster 268a includes the first cluster 264a and additional data points that were not included in the first cluster 264a as part of the first clustering tier. Similarly, the second cluster 268b includes the second cluster 264b and additional data points that were not included in the second cluster 264b as part of the first clustering tier.

As mentioned, and in certain embodiments, the different clustering tiers utilize different subsets of features and/or different subsets of parameters. Further, the values of the applicable parameters (i.e., thresholds) may be adjusted at any clustering tier to create clusters that include more or less similar data points than cluster sets formed during prior clustering tiers.

### Example Computer-Implemented Methods

FIG. 3 depicts a flow diagram representing an example computer-implemented method 300, in accordance with various embodiments described herein. The method 300 may be implemented by one or more processors of the example computing system 100, such as the processor 102a of central server 102, for example.

The method 300 includes receiving a plurality of data points that each include data corresponding to a feature set (block 302). The method 300 further includes applying a machine learning model to the plurality of data points, wherein applying the machine learning model includes: clustering, based on similarity values computed using a first subset of the feature set, a first portion of the plurality of data points into a first cluster set (block 304). The method 300 further includes applying a machine learning model to the plurality of data points, wherein applying the machine learning model includes: clustering, based on similarity values computed using a second subset of the feature set that is different from the first subset, a second portion of the plurality of data points into a second cluster set (block 306).

The method 300 further includes generating a data object indicating a course of action for an entity associated with a first data point based on the first data point being included in the first cluster set or the second cluster set (block 308). In some embodiments, the method 300 further includes determining that a respective data point of the plurality of data points is an un-clustered data point (optional block 310). In these embodiments, this identification may take place after clustering the second portion of the plurality of data points into the second cluster set. In certain embodiments, the method 300 further includes clustering the respective data point into a default cluster (optional block 312).

In certain embodiments, the machine learning model is a hierarchical clustering model, the first subset includes all features of the feature set, and the second subset includes fewer than all features of the feature set. In some embodiments, the similarity values are Jaccard distances.

In some embodiments, the feature set includes one or more of: (i) a co-morbidity value, (ii) a co-morbidity severity value, (iii) an age value, (iv) a gender value, (v) a procedure code, (vi) a diagnosis code, or (vii) a service location; and each feature of the feature set is a binary value or a one-hot encoded value.

In certain embodiments, the second portion of the plurality of data points and the first portion of the plurality of data points are mutually exclusive.

In some embodiments, clustering performed by the machine learning model (e.g., at blocks 304, 306) is further based on a parameter set that includes at least one of: (i) a data point neighbor value, (ii) a distance threshold, or (iii) a minimum cluster size.

Of course, it is to be appreciated that the actions of the method 300 may be performed any suitable number of times, and that the actions described in reference to the method 300 may be performed in any suitable order.

### EXAMPLES

Example 1. A computer-implemented method comprising: receiving, by one or more processors, a plurality of data points that each include data corresponding to a feature set; applying, by the one or more processors, a machine learning model to the plurality of data points, wherein applying the machine learning model includes: clustering, based on similarity values computed using a first subset of the feature set, a first portion of the plurality of data points into a first cluster set, and clustering, based on similarity values computed using a second subset of the feature set that is different from the first subset, a second portion of the plurality of data points into a second cluster set; and generating, by the one or more processors, a data object indicating a course of action for an entity associated with a first data point based on the first data point being included in the first cluster set or the second cluster set.

Example 2. The computer-implemented method of Example 1, wherein the machine learning model is a hierarchical clustering model.

Example 3. The computer-implemented method of any of Examples 1 or 2, wherein the similarity values are Jaccard distances.

Example 4. The computer-implemented method of any of Examples 1 through 3, further comprising: after clustering the second portion of the plurality of data points into the second cluster set, identifying, by the one or more processors, a respective data point of the plurality of data points is an un-clustered data point; and clustering, by the one or more processors, the respective data point into a default cluster.

Example 5. The computer-implemented method of any of Examples 1 through 4, wherein the first subset includes each feature of the feature set and the second subset includes less than each feature of the feature set.

Example 6. The computer-implemented method of any of Examples 1 through 5, wherein: the feature set comprises: (i) a co-morbidity value, (ii) a co-morbidity severity value, (iii) an age value, (iv) a gender value, (v) a procedure code, (vi) a diagnosis code, or (vii) a service location; and each feature of the feature set is a binary value or a one-hot encoded value.

Example 7. The computer-implemented method of any of Examples 1 through 6, wherein the second portion of the plurality of data points and the first portion of the plurality of data points are mutually exclusive.

Example 8. The computer-implemented method of any of Examples 1 through 7, wherein the first parameter set and the second parameter set further include at least one of: (i) a data point neighbor value, (ii) a distance threshold, or (iii) a minimum cluster size.

Example 9. A system comprising memory and one or more processors communicatively coupled to the memory, the one or more processors configured to: receive a plurality of data points that each include data corresponding to a feature set; apply a machine learning model to the plurality of data points, wherein applying the machine learning model includes: clustering, based on similarity values computed using a first subset of the feature set, a first portion of the plurality of data points into a first cluster set, and clustering, based on similarity values computed using a second subset of the feature set that is different from the first subset, a second portion of the plurality of data points into a second cluster set; and generate a data object indicating a course of action for an entity associated with a first data point based on the first data point being included in the first cluster set or the second cluster set.

Example 10. The system of Example 9, wherein the machine learning model is a hierarchical clustering model.

Example 11. The system of any of Examples 9 or 10, wherein the one or more processors are further configured to: after clustering the second portion of the plurality of data points into the second cluster set, identify a respective data point of the plurality of data points is an un-clustered data point; and cluster the respective data point into a default cluster.

Example 12. The system of any of Examples 9 through 11, wherein the first subset includes each feature of the feature set and the second subset includes less than each feature of the feature set.

Example 13. The system of any of Examples 9 through 12, wherein: the feature set comprises: (i) a co-morbidity value, (ii) a co-morbidity severity value, (iii) an age value, (iv) a gender value, (v) a procedure code, (vi) a diagnosis code, or (vii) a service location; and each feature of the feature set is a binary value or a one-hot encoded value.

Example 14. The system of any of Examples 9 through 13, wherein the second portion of the plurality of data points and the first portion of the plurality of data points are mutually exclusive.

Example 15. The system of any of Examples 9 through 14, wherein clustering performed by the machine learning model is further based on a parameter set that includes at least one of: (i) a data point neighbor value, (ii) a distance threshold, or (iii) a minimum cluster size.

Example 16. One or more non-transitory computer-readable storage media including instructions that, when executed by one or more processors, cause the one or more processors to: receive a plurality of data points that each include data corresponding to a feature set; apply a machine learning model, to the plurality of data points, wherein applying the machine learning model includes: clustering, based on similarity values computed using a first subset of the feature set, a first portion of the plurality of data points into a first cluster set, and clustering, based on similarity values computed using a second subset of the feature set that is different from the first subset, a second portion of the plurality of data points into a second cluster set; generate a data object indicating a course of action for an entity associated with a first data point based on the first data point being included in the first cluster set or the second cluster set.

Example 17. The one or more non-transitory computer-readable storage media of Example 16, wherein the machine learning model is a hierarchical clustering model.

Example 18. The one or more non-transitory computer-readable storage media of any of Examples 16 or 17, wherein the instructions further cause the one or more processors to: after clustering the second portion of the plurality of data points into the second cluster set, identify a respective data point of the plurality of data points is an un-clustered data point; and cluster the respective data point into a default cluster.

Example 19. The one or more non-transitory computer-readable storage media of any of Examples 16 through 18, wherein: the feature set comprises: (i) a co-morbidity value, (ii) a co-morbidity severity value, (iii) an age value, (iv) a gender value, (v) a procedure code, (vi) a diagnosis code, or (vii) a service location; each feature of the feature set is a binary value or a one-hot encoded value; and clustering performed by the machine learning model is further based on a parameter set that includes at least one of: (i) a data point neighbor value, (ii) a distance threshold, or (iii) a minimum cluster size.

Example 20. The one or more non-transitory computer-readable storage media of any of Examples 16 through 19, wherein the first subset includes each feature of the feature set and the second subset includes less than each feature of the feature set.

### ADDITIONAL CONSIDERATIONS

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

The systems and methods described herein are directed to an improvement to computer functionality, and improve the functioning of conventional computers. Additionally, certain embodiments are described herein as including logic or a number of routines, subroutines, applications, or instructions. These may constitute either software (e.g., code embodied on a non-transitory, machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules include a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based upon any statement made in any section of this patent (other than the language of the claims). To the extent that any term recited in the claims at the end of this disclosure is referred to in this disclosure in a manner consistent with a single meaning, that is done for sake of clarity only so as to not confuse the reader, and it is not intended that such claim term be limited, by implication or otherwise, to that single meaning.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also may include the plural unless it is obvious that it is meant otherwise.

Upon reading this disclosure, those of skill in the art will appreciate still additional alternative structural and functional designs for evaluation properties, through the principles disclosed herein. Therefore, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The patent claims at the end of this patent application are not intended to be construed under 35 U.S.C. § 112 (f) unless traditional means-plus-function language is expressly recited, such as “means for” or “step for” language being explicitly recited in the claim(s).

What is claimed is:

1. A computer-implemented method comprising:
receiving, by one or more processors, a plurality of data points that each include data corresponding to a feature set;
applying, by the one or more processors, a machine learning model to the plurality of data points, wherein applying the machine learning model includes:
clustering, based on similarity values computed using a first subset of the feature set, a first portion of the plurality of data points into a first cluster set, and
clustering, based on similarity values computed using a second subset of the feature set that is different from the first subset, a second portion of the plurality of data points into a second cluster set; and
generating, by the one or more processors, a data object indicating a course of action for an entity associated with a first data point based on the first data point being included in the first cluster set or the second cluster set.

2. The computer-implemented method of claim 1, wherein the machine learning model is a hierarchical clustering model.

3. The computer-implemented method of claim 1, wherein the similarity values are Jaccard distances.

4. The computer-implemented method of claim 1, further comprising:
after clustering the second portion of the plurality of data points into the second cluster set, determining, by the one or more processors, that a respective data point of the plurality of data points is an un-clustered data point; and
clustering, by the one or more processors, the respective data point into a default cluster.

5. The computer-implemented method of claim 1, wherein the first subset includes all features of the feature set and the second subset includes fewer than all features of the feature set.

6. The computer-implemented method of claim 1, wherein:
the feature set comprises one or more of: (i) a co-morbidity value, (ii) a co-morbidity severity value, (iii) an age value, (iv) a gender value, (v) a procedure code, (vi) a diagnosis code, or (vii) a service location; and
each feature of the feature set is a binary value or a one-hot encoded value.

7. The computer-implemented method of claim 1, wherein the second portion of the plurality of data points and the first portion of the plurality of data points are mutually exclusive.

8. The computer-implemented method of claim 1, wherein clustering performed by the machine learning model is further based on a parameter set that includes at least one of: (i) a data point neighbor value, (ii) a distance threshold, or (iii) a minimum cluster size.

9. A system comprising memory and one or more processors communicatively coupled to the memory, the one or more processors configured to:
receive a plurality of data points that each include data corresponding to a feature set;
apply a machine learning model to the plurality of data points, wherein applying the machine learning model includes:
clustering, based on similarity values computed using a first subset of the feature set, a first portion of the plurality of data points into a first cluster set, and
clustering, based on similarity values computed using a second subset of the feature set that is different from the first subset, a second portion of the plurality of data points into a second cluster set; and
generate a data object indicating a course of action for an entity associated with a first data point based on the first data point being included in the first cluster set or the second cluster set.

10. The system of claim 9, wherein the machine learning model is a hierarchical clustering model.

11. The system of claim 9, wherein the one or more processors are further configured to:
after clustering the second portion of the plurality of data points into the second cluster set, determine that a respective data point of the plurality of data points is an un-clustered data point; and
cluster the respective data point into a default cluster.

12. The system of claim 9, wherein the first subset includes all features of the feature set, and the second subset includes fewer than all features of the feature set.

13. The system of claim 9, wherein:
the feature set comprises one or more of: (i) a co-morbidity value, (ii) a co-morbidity severity value, (iii) an age value, (iv) a gender value, (v) a procedure code, (vi) a diagnosis code, or (vii) a service location; and
each feature of the feature set is a binary value or a one-hot encoded value.

14. The system of claim 9, wherein the second portion of the plurality of data points and the first portion of the plurality of data points are mutually exclusive.

15. The system of claim 9, wherein clustering performed by the machine learning model is further based on a parameter set that includes at least one of: (i) a data point neighbor value, (ii) a distance threshold, or (iii) a minimum cluster size.

16. One or more non-transitory computer-readable storage media including instructions that, when executed by one or more processors, cause the one or more processors to:
receive a plurality of data points that each include data corresponding to a feature set;
apply a machine learning model, to the plurality of data points, wherein applying the machine learning model includes:
clustering, based on similarity values computed using a first subset of the feature set, a first portion of the plurality of data points into a first cluster set, and
clustering, based on similarity values computed using a second subset of the feature set that is different from the first subset, a second portion of the plurality of data points into a second cluster set; and
generate a data object indicating a course of action for an entity associated with a first data point based on the first data point being included in the first cluster set or the second cluster set.

17. The one or more non-transitory computer-readable storage media of claim 16,
wherein the machine learning model is a hierarchical clustering model.

18. The one or more non-transitory computer-readable storage media of claim 16, wherein the instructions further cause the one or more processors to:
after clustering the second portion of the plurality of data points into the second cluster set, determine that a respective data point of the plurality of data points is an un-clustered data point; and
cluster the respective data point into a default cluster.

19. The one or more non-transitory computer-readable storage media of claim 16, wherein:
the feature set comprises one or more of: (i) a co-morbidity value, (ii) a co-morbidity severity value, (iii) an age value, (iv) a gender value, (v) a procedure code, (vi) a diagnosis code, or (vii) a service location;
each feature of the feature set is a binary value or a one-hot encoded value; and
clustering performed by the machine learning model is further based on a parameter set that includes at least one of: (i) a data point neighbor value, (ii) a distance threshold, or (iii) a minimum cluster size.

20. The one or more non-transitory computer-readable storage media of claim 16, wherein the first subset includes all features of the feature set and the second subset includes fewer than all features of the feature set.
